(19) United States
(12) Plant Patent Application Publication
(10) Pub. No.: US 2002/0162147 P1
(43) Pub. Date: Oct. 31, 2002

(54) PEACH TREE NAMED 'SUPECHFOURTEEN'

(76) Inventors: Carlos D. Fear, Aptos, CA (US); Bruce D. Mowrey, La Selva Beach, CA (US); David W. Cain, Bakersfield, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/792,850

(22) Filed: Feb. 23, 2001

(51) Int. Cl. A01H 5/00

(52) U.S. Cl. PLT/190

(57) ABSTRACT

A new and distinct peach variety that possesses a larger-sized fruit. The fruit of the new variety has consistently firmer fruit flesh.

### BACKGROUND AND SUMMARY OF THE INVENTION

This invention relates to the discovery and asexual propagation of a new variety of peach, Prunus persica cv. ‘Supechfourteen.’ The new variety was first hybridized by Carlos D. Fear on May 20, 1991. The new variety cv. ‘Supechfourteen’ was selected and evaluated by Bruce D. Mowrey and David W. Cain near Wasco, Kern County, Calif. The variety was originated by hybridization. The new variety cv. ‘Supechfourteen’ is characterized by producing larger, consistently firmer fruits with high levels of external red blush.

The seed parent is ‘Flordaking’ (unpatented) and the pollen parent is ‘Flordaprince’ (unpatented). The parent varieties were first crossed February 1988. The date of sowing was October 1988, while the first flowering occurred in February, 1991. The new ‘Supechfourteen’ variety was first asexually propagated by Bruce D. Mowrey in June 1991, near Wasco, Kern County, Calif., by budding. The new peach variety ‘Supechfourteen’ ripens at approximately the same time as its pollen parent ‘Flordaprince’ (unpatented). It differs from ‘Flordaprince’ by producing consistently larger-sized fruit and firmer fruit flesh.

The new peach variety ‘Supechfourteen’ may be distinguished from its seed parent ‘Flordaking’ (unpatented) by having a higher level of external red blush than ‘Flordaking’ (70-90% as compared to 50-70% with ‘Flordaking’). Fruits of ‘Supechfourteen’ are firmer, rounder, and have fewer split stones than ‘Flordaking’. Moreover, the new cultivar has a lower winter chilling requirement of approximately 150 hours of temperatures below 45° F. as compared to 450 hours required for ‘Flordaking’.

The new peach variety cv. ‘Supechfourteen’ ripens approximately 10 days before the ‘Tropic Beauty’ variety.

The new peach variety cv. ‘Supechfourteen’ has been shown to maintain its distinguishing characteristics through successive asexual propagations by, for example, budding.

### BRIEF DESCRIPTION OF THE FIGURE

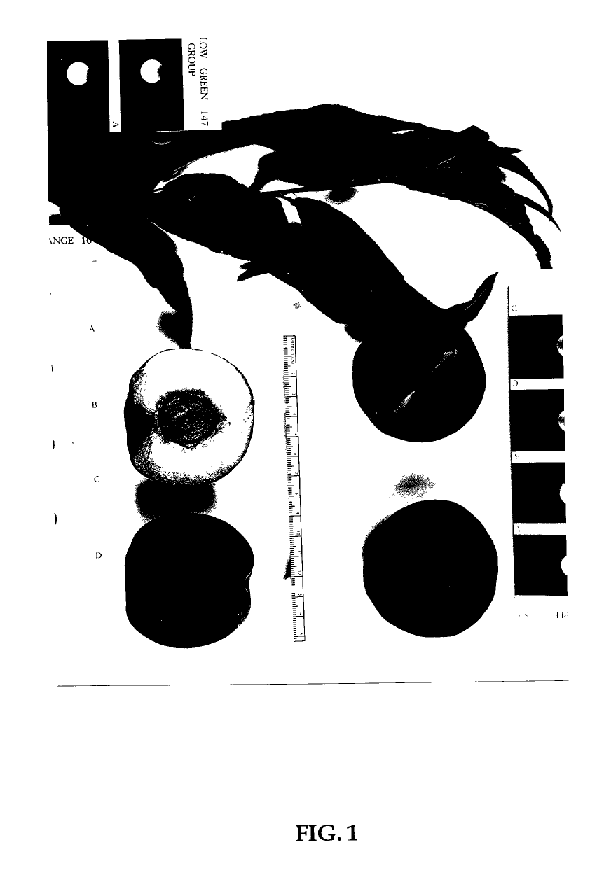

The accompanying drawing in FIG. 1 illustrates in full color.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon the R.H.S. Colour Chart, published by The Royal Horticultural Society, London, England.

The descriptive matter which follows pertains to ‘Supechfourteen’ plants grown in the vicinity near Wasco, Kern County, Calif., during 2000, and is believed to apply to plants of the same variety grown under similar conditions of soil and climate elsewhere:

### TREE

General: Trees were five years old and were maintained at a height of about ten feet and a canopy diameter of about ten feet by annual prunings.

Size.—medium.

Vigor.—medium.

Habit.—semi-upright.

Density of Foliage.—medium.

Shape.—vase formed.

Hardiness.—hardy near Wasco, Calif.

Productivity.—productive.

Fruit bearing.—regular bearer.

Root stock.—Nemared.

Trunk: Trunk is round in outline and of medium circumference, averaging 71 cm at 30 cm above ground level on 5-year-old trees.

Shape.—medium.

Surface texture.—smooth—medium.

Color.—about 199B.

Branches: Branches are round in outline and average 27 cm in circumference on 5-year-old trees as measured 30 cm from the branch juncture with the trunk.

Shape.—medium—slender.

Surface texture.—smooth—medium.

Surface appearance.—semi-glossy—dull.

Lenticels:

Number.—medium—few.

Size.—large—medium.

Density.—1.6/cm²

### LEAVES

General:

Average length.—about 17 cm.

Average Width.—about 4 cm.

Size.—medium.

Outline.—lanceolate.

Profile.—up folded.

Leaf blade tip.—curved downwardly.

Angle of tip.—acute.

Margin.—finely serrate.

Undulation of margin.—slight.

Apex.—acuminate.

Base.—U-shaped.

Thickness.—medium.

Upper Surface:

Color.—about Yellow-Green group 147A.

Glossiness.—medium.

Pubescence.—absent.

Surface texture.—rugose.

Surface appearance.—semi-glossy.

Lower Surface:

Color.—about Yellow-Green group 147B.

Glossiness.—weak.

Pubescence.—absent.

Surface texture.—rugose.

Surface appearance.—dull.

Petiole:

Length.—medium—short, about 1.1 cm.

Thickness.—thick—medium, about 2 mm.

Glands:

Average number.—3.6.

Positioning.—on both leaf base and petiole.

Size.—medium.

Shape.—reniform.

Color.—about Yellow-Green group 153D.

Stipules:

Persistence.—fall off.

Wood (Leaf) Buds:

Shape.—ovoid.

Size.—small—medium.

Position relative to shoot.—adpressed.

Support.—decurrent.

Size.—small.

Time of bud burst.—medium.

### FLOWERING SHOOTS:

Anthocyanin coloration.—present.

Intensity.—medium.

Thickness.—thin—medium, about 0.4 cm.

Internode length.—medium, about 3 cm midway on flowering shoots.

Density of buds.—medium.

Distribution.—on one-year-old shoots.

Ratio of wood (leaf) buds to flowering shoots.—1:2.5.

### FLOWERS

Flower-Buds:

Hardiness.—hardy under central California conditions.

Size.—medium.

Length.—medium.

Shape.—plump.

Positioning.—free.

Pubescence.—pubescent.

Color.—about Greyed-Orange group 165A.

General:

Date of first bloom.—Jan. 1, 2000 at a low chill site near Mecca, Riverside County, Calif.; Feb. 2, 20000 at a medium-high chill site near Wasco, Kern County, Calif.

Date of full bloom.—Jan. 1, 2000 at a low chill site near Mecca, Riverside County, Calif.; Feb. 8, 2000 at a medium-high chill site near Wasco, Kern County, Calif.

Time of bloom.—medium, as compared with similar varieties in the growing area of Mecca, Riverside County, Calif.

Duration of bloom.—medium, about 10 days.

Size (diameter of the fully open flower).—large, about 4.8 cm.

Shape.—rosaceous.

Petals.—overlapping.

Color (fully opened flower): about 62D.

Peduncle:

Length.—short.

Thickness.—medium.

Pubescence.—absent.

Receptacle:

Depth.—medium.

Pubescence of inner surface (at white bud stage).—absent.

Pubescence of outer surface.—absent.

Sepals:

Positioning.—adpressed to petals.

Shape.—ovate.

Pubescence of inner surface.—absent.

Pubescence of outer surface.—medium.

Frequency of flowers with double sepals.—none.

Petals:

Frequency of flowers with double petals.—none.

Size.—medium, about 1.6 cm. width.

Shape.—transverse broad elliptic.

Claw length.—medium.

Margin waviness.—medium.

Base angle.—medium.

Division of upper margin.—entire.

Pubescence of inner surface.—absent.

Pubescence of outer surface.—absent.

Color (inner surface).—about 62D.

Color (outer surface).—about 62D.

Stigma:

Position (as compared with anthers).—level.

Anthers:

Color (just before dehiscence).—about Red 46D.

Pollen:

Color.—about 14B.

Stamens:

Position.—perigynous.

Pistil:

Frequency of supplementary pistils.—few.

Number.—sometimes more than one.

Ovary:

Pubescence.—pubescent.

Density of hairs.—medium.

Style:

Pubescence (of base).—medium.

Density of hairs.—slight.

### FRUIT

General:

Maturity when described.—firm—ripe.

Date.—Apr. 15, 2000 at the low-chill site near Mecca, Riverside County, Calif.; 5/8/00 at the medium-high chill site near Wasco, Kern County, Calif.

Size.—slightly variable—large; about 6.5 cm, 135 g.

Axial diameter.—about 6 cm.

Transverse in suture plane.—about 6.5 cm.

At right angles to suture plane.—about 6.5 cm.

Form.—uniform—globose.

Position of maximum diameter.—towards stem end.

Symmetry about the suture.—slightly asymmetric.

Season ripening.—early.

Use.—market.

Keeping quality.—medium.

Resistance to.—Insects: good. Diseases: good. Shipping quality: medium.

Suture: an inconspicuous line, has slight-marked depression beyond pistil point.

Ventral Surface:

Shape.—rounded slightly.

Lips.—equal.

Depression of apex.—distinct.

Pistil base.—not persisting.

Pubescence at apex.—medium.

Stem Cavity:

Shape.—flaring—circular; with suture showing on one side.

Depth.—about 1.5 cm.

Breadth.—about 2.8 cm.

Base:

Shape.—rounded.

Apex:

Shape.—slightly depressed.

Pistil Point:

Shape.—slightly depressed.

Stem:

Length.—stout—glabrous, about 0.8 cm.

Adherence to stone.—strong.

Skin:

Thickness.—medium.

Texture.—medium.

Reticulation.—absent.

Roughness.—absent.

Tenacity.—tenacious to flesh.

Tendency to crack.—none in wet season.

Color.—Ground color: 1B; Overcolor: 59A.

Down (pubescence).—moderate to scant—short.

Flesh:

Color.—about 12C midway between pit and skin; about 1C adjacent to the pit cavity.

Amygdalin.—wanting.

Juice.—moderate.

Sugar content.—low—medium.

Texture.—medium, coarse, melting.

Fibres.—abundant, coarse.

Ripens.—earliest at apex.

Flavor.—delicate.

Eating quality.—fair—good, for early season fruit.

Stone/flesh ratio.—1 cm diameter: 3 cm diameter.

### STONE

General:

Adherence to flesh.—cling, adheres to flesh over entire surface.

Fibres.—long.

Size.—medium.

Length.—about 3.2 cm.

Breadth.—about 1.8 cm to about 2.2 cm.

Width of stalk end.—about 0.4 cm.

Angle of stalk end.—obtuse.

Form (profile).—oval.

Form (ventral view).—globular.

Base.—oblique.

Hilum.—oval.

Apex.—pointed.

Position of maximum breadth.—middle.

Sides.—equal.

Surface.—regularly firrowed near base, pitted throughout.

Outgrowing keel.—partly developed.

Ridges.—rounded towards base.

Pits.—elongated.

Ventrical edge.—thin with slight wing toward base.

Dorsal edge.—narrow.

Color of stone.—about 165D Greyed-Orange group.

Tendency to split.—slight in wet season.

Supechfourteen: Table of R.H.S. Color Codes

| | | | R.H.S. |
|---|---|---|---|
| | | | Color Code |
| TREE: | Trunk: | | 199B |
| LEAVES: | Upper Surface: | | 147A |
| | Lower Surface: | | 147B |
| | Glands: | | 153D |
| FLOWERS: | Flower Buds: | | 165A |
| | General: | Fully opened flower: | 62D |
| | Petals: | Inner surface: | 62D |
| | | Outer surface: | 62D |
| | Anthers: | Just before dehiscence: | 46D |
| | Pollen: | | 14B |
| FRUIT: | Skin: | Ground color: | 1B |
| | | Overcolor: | 59A |
| | Flesh: | Midway between pit and | 12C |
| | | skin: | |
| | | Adjacent to the pit cavity: | 1C |
| STONE: | | Stone color: | 165D |

What is claimed is:

1. A new and distinct variety of peach cv. ‘Supechfourteen’ as herein illustrated and described.
